(19) United States
(12) Patent Application Publication
(10) Pub. No.: US 2003/0032597 A1
(43) Pub. Date: Feb. 13, 2003

(54) Targeting nucleic acids to a cellular nucleus

(76) Inventor: Magdolna G. Sebestyen, Madison, WA (US)

Correspondence Address: Mark K. Johnson, Mirus, 505 South Rosa Road, Madison, WI, 53719, US

(21) Appl. No.: 10/200,800

(22) Filed: Jul. 22, 2002

Related U.S. Application Data
(60) Provisional application No. 60/309,319, filed on Jul. 31, 2001.

(51) Int. Cl. A61K 48/00; A61K 38/17

(52) U.S. Cl. 514/12; 514/44

(57) ABSTRACT

We disclose gene delivery systems that target exogenous nucleic acids to the nucleus of mammalian cells and are delivered to chromatin during cellular mitosis, remaining within the nucleus after mitosis.

This invention relates to gene delivery systems which target exogenous nucleic acids to the nucleus of actively dividing mammalian cells during mitosis.

### BACKGROUND

All eukaryotic cells are divided into functionally distinct, membrane-bound compartments. The two major compartments pertinent to gene delivery are the cytoplasm and the nucleus. Most of the currently used non-viral gene delivery methods deposit the DNA into the cytoplasm, from where it must be further transported to the nucleus, where transcription can take place. The two compartments are separated by the nuclear envelope (NE): two concentric membrane layers punctured by pores. The pores, called nuclear pore complexes (NPCs), are formed by supramolecular assemblies of multiple copies of some 30-50 different proteins¹. NPCs allow the selective, active transport of macromolecules in both directions across the nuclear envelope provided they carry specific signals, or addresses, called nuclear localizing signals (NLS) and nuclear export signals (NES). These signals are recognized by receptor molecules, which in turn mediate translocation through the central channel of the pore^{2,3}. Macromolecules larger than 50-60 kDa cannot efficiently cross the nuclear envelope without displaying such signals.

High transfection efficiencies, up to 100%, would be extremely beneficial for several research applications as well as for in vivo gene therapy. However, current methods to transfect genes into cultured cells with high efficiency often involve the use of viral vectors or are associated with high levels of toxicity. Viral gene delivery is likely to increase the chance of rejection after transfer due to display of viral antigens. High toxicity is associated with electroporation and high doses of cationic lipids. While these parameters may be acceptable for some in vitro applications, they are incompatible with many other in vitro applications and all in vivo gene therapy usage.

Most of the currently used non-viral gene delivery methods deposit the DNA into the cytoplasm. From there it must be transported to the nucleus in order for expression to occur. Thus, one of the major physical barriers for effective delivery of plasmid DNA (pDNA) into mammalian cells is the nuclear envelope. It is believed that the breakdown and reassembly of the nuclear envelope during mitosis allows entry of DNA into the nucleus and accounts for improved transfection efficiencies observed in dividing cells⁴⁻⁶. In fact, for some oncoretroviruses (e.g. MLV), whose preintegration complexes are unable to be transported through the NPC of the intact interphase nuclear envelope, nuclear entry depends on the breakdown of the nuclear membrane at the onset of mitosis. However, the disassembly of the NE alone is insufficient to ensure that the preintegration complex will partition to a newly formed nucleus at the end of mitosis. These viruses possess mechanisms to enhance retention of their genomes in the nucleus^{6,7}. Similarly, disassembly of the NE during mitosis results only in a very limited increase in expression of transfected genes. Studying the sub-cellular distribution of macromolecules after mitosis we have shown that pDNA and large dextran are mostly excluded from the re-forming nuclei. The molecular details of nuclear assembly at the end of mitosis suggest that only the chromosomes and molecules physically associated with them become enclosed within the new nucleus as the envelope forms closely around the chromatin¹¹. We postulate that this strict sorting mechanism is one of the reasons why marker gene expression efficiency remains far below 100%, even in actively dividing cultured cells.

Two conceptually different pathways can be used to accomplish the nuclear targeting of exogenous DNA in mitotic cells. First, the traditional nuclear localization signal (NLS) mediated process can theoretically promote the transport of pDNA molecules through NPCs. All published efforts for the enhancement of gene delivery to the nucleus have focused on this method. A variety of such NLS signals have been used in attempts to target exogenous DNA to the interphase nucleus¹⁰⁻¹². We suspect that, like endogenous nuclear proteins, NLS-labeled DNA transported into the nucleus during interphase becomes excluded again from nuclei at the end of mitosis. The second method, proposed in this invention, describes associating a biologically active compound with mitotic components to increase the efficiency of nuclear uptake and retention of the biologically active compound in dividing cells.

### BRIEF DESCRIPTION OF FIGURES

FIG. 1. Sub-cellular location of 500 kDa dextran (lower left panel; A, B, C, D) and Cy5-labeled plasmid DNA (upper left panel; A, B, C, D) in undivided (A, C) or divided (B, D) cells 16-22 h after delivery into either the cytoplasm (A, B), or nucleus (C, D). The majority of cells divided by this time, but a small population of undivided cells remained. The EYFP-Nuc protein, encoded by the injected plasmid DNA, emits green fluorescence (upper right panel; B, C, D) and predominantly accumulates in nucleoli. In contrast, both the dextran and the DNA are excluded from the nucleoli of the undivided cell after nuclear delivery (C). In divided cells, both dextran and DNA are excluded from nuclei of the daughter cells independent of whether they were injected into the cytoplasm or the nucleus (D). The image in the bottom right corner of each panel is the merged image of all three channels. Images were collected using confocal microscopy. Each image represents a single 0.5 μm optical section.

FIG. 2. Distribution of the Ki-67 antigen at different stages of the cell cycle. Synchronized HeLa cells were probed with anti-Ki-67 MAb. Alexa488-anti Mouse IgG (upper panel) and ToPro3 DNA staining (lower panel) are shown.

FIG. 3. Sub-cellular distribution of various Ki-67 domains expressed as EYFP-fusions in transiently transfected HeLa cells. EYFP-Ki fusion protein (upper panel; Interphase, Mitosis); ToPro3 DNA staining (lower panel; Interphase, Mitosis).

### SUMMARY

In a preferred embodiment, we describe a process to increase targeting of a biologically active compound to the nucleus of a dividing cell as the cell proceeds through mitosis comprising associating the compound with a Chromosome Targeting Signal(CTS). This targeting signal is distinct from the traditional nuclear localization sequence (NLS), in that it does not initiate the transport of the compound into interphase nuclei through nuclear pore complexes (NPCs). Rather, the CTS targets the cargo to which it is associated to the chromosomes during mitosis, resulting in enhanced localization within the re-assembled nuclei. A preferred biologically active compound is a nucleic acid or a nucleic acid complex. Another preferred biologically active compound is a protein or drug that exerts its effect in the nucleus but is unable to enter an interphase nucleus through NPCs. The CTS may be used to enhance nuclear localization of a compound in a cell that is in vivo or in vitro.

In a preferred embodiment, we describe a process for associating a biologically active compound with mitotic chromosomes resulting in partitioning of the compound to the nuclear compartment prior to the end of telophase. The CTS may be used to enhance nuclear localization of a compound in a cell that is in vivo or in vitro.

In a preferred embodiment, the CTS is used to prolong residence of the biologically active compound in the nucleus in dividing cells. During reformation of the nuclear envelope at the end of mitosis, most compounds not associated with chromosomes are excluded from the newly formed nucleus. Without a functional NLS, these compounds do not gain re-entry into the nucleus. Association of a compound with a CTS would increase its retention in the nucleus as a cell progresses through mitosis. The cell my be in vivo or it may be in vitro.

In a preferred embodiment, any chromatin-associating compound that associates with mitotic chromosomes and is incorporated into newly formed nuclei at the end of mitosis can potentially serve as a Chromosome Targeting Signal (CTS). Components of chromosomal structures present in or on chromatin either constitutively or during mitosis before the onset of telophase can potentially be used as CTSs.

Proteins that may serve a chromosomal targeting signal may be selected from the group comprising:

1. Proteins associated with nuclear envelope precursor vesicles.

2. Structural proteins of the chromosomes or chromatin; including histone H1, histone H2a, histone H2b, histone H3, histone H4, and Topoisomerase II.

3. Proteins that are natural components of anaphase/telophase chromatin. These proteins may be constitutive structural elements or may be present on the chromosomes specifically during this period of the cell cycle.

4. Nucleolar proteins; including nucleolin, peripherin, Topoisomerase 1, Fibrillarin, etc.

5. Nucleoskeletal proteins; including lamin B1 and B2, etc.

6. Structural proteins of the kinetochore, the large multi-protein complex on the centromere of each chromosome. Proteins that have been identified in the kinetochore include: mitosin, CENP-B, CENP-C, CENP-D, CENP-E, CENP-F, CENP-G, CENP-H, INCENP, MCAK, ZW10.

7. Chromatin binding domains of histone modifying enzymes including: histone deacetylases, histone acetyltransferases, histone methyltransferases, histone kinases, histone dephosphorylases.

8. Binding domains of other chromatin-regulatory proteins, including bromodomain proteins, and chromodomain proteins.

9. Histone associating proteins.

10. CENP-A; a centromere specific histone protein.

11. Lamin B1; The C-terminal domain, residues 372-586, contains a putative NLS, and a long stretch of acidic residues close to the C-terminus, which is thought to be responsible for chromatin binding.

12. LBR; the lamin B receptor protein, a chromatin and lamin binding protein from the inner nuclear membrane¹³. Chromatin binding domain mapped to amino acid residues 97-174¹⁴.

13. LEM domain proteins.

14. Lamina associated protein (LAP) family members; isoforms of the lamina associated protein family including: LAP1, LAP2a, LAP2β, LAP2? and LAP2d isoforms.

15. LAP2a; lamina associated polypeptide 2 alpha isoform, also called thymopoietin alpha.

residues 270-615, unique domain in the LAP2a shown to intiate binding to the anaphase chromosomes during early stages of nuclear re-assembly¹⁵.

residues 1-188, conserved chromatin-binding domain of all LAP2 isoforms

residues 189-615, unique domain of LAP2a with a putative NLS on the N-terminus.

16. LAP2β; lamina associated polypeptide 2 beta isoform, also called thymopoietin beta.

BAF-binding region without the transmembrane domains.

residues 1-188, domain common to all LAP2 isoforms.

residues 1-408, domain that interacts with LMNB1

17. Emerin; an integral protein of the inner nuclear membrane. GFP-tagged emerin accumulated on chromosomes 5 minutes after the onset of anaphase¹⁶.

18. MAN1; shares a homologous domain (LEM module) with LAP2β.

19. HP1; Heterochromatin protein 1, a non histone chromosomal protein.

20. NUP153¹⁶.

21. Nurim; a nuclear envelope membrane protein, which is very tightly associated with the nucleus¹⁷.

22. NEP-B78; which may be required for the targeting of nuclear envelope vesicles to the surface of decondensing chromatin¹⁸.

23. BAF; barrier to autointegration factor, whose cellular functions may include the establishment of higher order chromatin structure, and to which LAP2β binds.

24. Condensin; highly conserved multi-protein complex belonging to the SMC (structural maintenance of chromosomes) family that is distinctly chromosomally bound during mitosis. Its chromatin-binding elements (e.g. CNAP-1) are chromosome targeting signals¹⁹.

25. hCAP-C/hCAP-E; human chromosome associated protein -C and -E complex is required for mitotic chromosome condensation.

26. RCC1; regulator of chromosome condensation protein, also called RanGEF (Ran guanine nucleotide exchange factor).

27. NuMa; nuclear mitotic apparatus proteins, a group of 200-240 kDa non-histone proteins common in mammalian cells. It has been shown to directly associate with condensed telophase chromosomes earlier than the association of lamins can be detected²⁰.

28. hMCM4p; DNA replication factor that binds to chromatin during late anaphase. Mouse mcm2 binds to histone. Amino acid residues 63-153 are responsible for this binding²¹.

29. SUV39H1; suppressor of position effect variegation homologue, which transiently accumulates at centromeric positions on the chromosomes during mitosis.

30. Ki-67; the C-terminal domain of this protein (KiF, residues 2937-3256) was shown in our preliminary studies to bind mitotic chromosomes.

31. Otefin and lamin isoforms Dm1 and Dm2, and their human homologues; required for chromatin binding of nuclear envelope precursor vesicles in Drosophila.

32. ATRX; localized to pericentromeric heterochromatin during interphase and mitosis. ATRX contains a highly conserved plant homeodomain (PHD)-like domain, present in many chromatin-associated proteins. The isolated PHD-like domain itself is also a potential targeting signal.

33. AKAP95; A-kinase anchoring protein, is associated with the nuclear matrix in interphase and redistributes mostly into a chromatin fraction at mitosis.

34. HA95; tightly associated with chromatin and the nuclear matrix/lamina network in interphase, and is bound to chromatin at mitosis.

35. TTF-1; colocalizes with the inactive transcription machinery present in certain mitotic nucleolar organizer regions (NORs).

36. UBF; DNA-binding transcription factor, remains strongly bound to rDNA loci on chromosomes during mitosis.

37. KLP38B; kinesin-related protein that colocalizes with condensed chromatin during cell division.

38. Rad17p; chromatin associated throughout the cell cycle.

39. p120; prototypic member of a growing subfamily of Armadillo-domain proteins found at cell-cell junctions and in nuclei

40. Mitotic Chromosomal Autoantigens (MCAs).

41. PNUTS, a putative protein phosphatase 1 nuclear targeting subunit, which co-localizes with the chromosomes during telophase.

42. VP22; Herpes simplex virus (HSV) tegument protein. During mitosis the protein enters the nucleus by binding to the mitotic chromosomes.

43. LANA (LNA1); Latency-associated nuclear antigen 1, another HSV protein able to associate with mitotic chromosomes⁷.

44. EBNA1; Epstein-Barr Virus (EBV) that binds to metaphase chromosomes⁸.

45. Viral proteins responsible for the nuclear targeting and long-term maintenance of the viral genome in the host cell's nucleus.

In a preferred embodiment, any protein that interacts with any of the above listed potential CTSs may be a CTS. In another preferred embodiment, any protein that is homologous to any of the above listed potential CTSs may be a CTS. In another preferred embodiment, any compound that interacts with any of the above listed potential CTSs may be a CTS. In another preferred embodiment, any recombinant protein, protein fragment of any of the above listed potential CTSs may be a CTS. The CTS may also be a synthetic peptide that has sequence similar to a portion of any of the above proteins. In another preferred embodiment, any antibody or antibody fragment that interacts with any of the above listed potential CTSs may be a CTS.

In a preferred embodiment, antibodies to components of chromosomal structures present in the chromatin either constitutively or during mitosis before the onset of telophase may be used as CTSs. Antibodies binding to any of the proteins accessible on the surface of anaphase chromosomes are potential CTSs. In another preferred embodiment, antibodies against mitotic chromosomal autoantigens (MCAs) may be used as CTSs. MCAs are identified by autoimmune sera exclusively on mitotic chromosome arms, with no staining in interphase nuclei²². In another preferred embodiment, antibodies against members of the nuclear hormone receptor superfamily may me used as CTSs. Nuclear hormone receptors recruit large protein complexes to the chromatin to reversibly stabilize or destabilize the chromatin, thereby affecting gene expression. Many components of these multi-subunit factors can be considered for this approach (e.g. CRSP, NAT, ARC, DRIP, VP16, p65, SREBP-1a etc.).

In a preferred embodiment, any synthetic or natural peptide or compound that interacts with chromosomes and is incorporated into newly from nuclei at the end of mitosis may be a CTS.

In a preferred embodiment, the CTS is associated with or attached to a molecule by a covalent linkage. The linkage may or may not include a spacer group. The linkage also may or may not include a labile or reversible bond.

In another preferred embodiment, the CTS is associated with or attached to a molecule by a non-covalent linkage. As an example, the CTS is attached to the protein streptavidin and biotin is linked to biologically active compound. The CTS is then associated with the biologically active compound through the streptavidin-biotin interaction. Antibody-epitope interaction is another method of non-covalently linking the CTS to a molecule

In a preferred embodiment, the CTS is linked to a compound or compounds, such as a transfection reagent, which is formed into a complex with a biologically active compound. The biologically active compound may be a nucleic acid. The CTS may be attached to a polymer such as Histone Hi protein, poly-ethylenimine, or poly-lysine. The CTS may be attached to an amphipathic compound such as a lipid. After delivery of the biologically active compound complex to an animal cell, the CTS enhances nuclear localization of the biologically active compound during mitosis. The attachment may be covalent or non-covalent. The attachment may or may not include a linker or spacer group. The attachment also may or may not include a labile or reversible bond.

In a preferred embodiment, microinjection of CTS-tagged DNA into the pronuclei of an egg could be used to increase the success rate of generating transgenic animals. Since integration of the transgene into the host cell's chromosome frequently does not occur before the initial cell division, the addition of a CTS would increase the amount of transgene DNA taken into the nuclei of the early embryo cells during the initial divisions.

In a preferred embodiment, the CTS may be used in combination with other functional groups or signals. These signals include cell targeting signals, nuclear localization signals, membrane active compounds, etc, and may aid in targeting the biologically active compound to specific cells types, binding to cell receptors to aid in internalization, enhancing escape from membrane enclosed compartments such as endosomes or avoidin undesirable interaction such as with serum components.

In a preferred embodiment, the CTS can be used to deliver a toxic compound to an actively dividing cell such as a cancer cell. The toxic compound can be a nucleic acid that encodes a suicide gene. Expression of the suicide gene in the actively dividing cell would kill the cell.

Further objects, features, and advantages of the invention will be apparent from the following detailed description when taken in conjunction with the accompanying drawings.

### DETAILED DESCRIPTION

Several research groups have reported that mitosis enhances marker gene expression, believed to be aided by the breakdown of the nuclear envelope (NE) during cell division. However, even after delivery of large amounts of DNA directly into the cytoplasm, significantly less than 100% of cells express the injected gene following mitosis²³. When examining the localization of cytoplasmically microinjected fluorescent DNA in HeLa cells, we observed essentially all the DNA in the cytoplasm, even in cells expressing the encoded marker gene. Furthermore, we have observed the exclusion of fluorescent pDNA from the re forming nuclei after mitosis, suggesting that mitosis itself fails to provide free access to the nuclear compartment. Our hypothesis is that a strict sorting mechanism of bona fide nuclear components inhibits the nuclear partitioning of non-chromatin molecules, such as exogenous pDNA, even if the pDNA had entered the nucleus prior to mitosis. Therefore, association of a compound with chromatin during mitosis will enhance nuclear localization of the compound. This hypothesis is supported by recent studies on the disassembly and reassembly of the nucleus during mitosis.

In all higher eukaryotic cells the NE temporarily breaks down during mitosis enabling components normally confined to the cytoplasm to interact with components of the nucleus. At the end of anaphase NE-specific proteins accumulate in membrane patches that are in contact with the surface of the chromosomes. These patches expand and, during telophase at the end of mitosis, fuse along the surface of the chromosomes leaving very little free aqueous volume trapped inside^{13,24-26}. This process effectively excludes molecules that are not tightly associated with the chromosomes from being included within the newly formed nuclei.

The nuclear lamina provides structural support for the NE as well as attachment sites for components of the chromatin. Like the NE, the lamina also disassembles at the onset of mitosis, and both its major constituents, the A-type and B-type lamin isoforms, show diffuse cytoplasmic staining. During anaphase lamin B1 (LMNB1) starts to accumulate on the surface of the chromosomes, followed by a rapid process of enclosing the entire perimeter of the region containing the chromosomes.

The major players of membrane recruitment to the surface of the chromatin are the lamin B receptor (LBR), members of the lamina-associated polypeptide (LAP) family, emerin, MAN1, and nurim^{16,17}. These proteins are all anchored to the inner layer of the NE, carry chromatin binding and/or lamin B binding motifs, and co-localize to the periphery of the chromosomes during late anaphase and early telophase.

During mitosis, nuclear matrix and nucleolar components form a dense peri-chromosomal sheath, which is present on every chromosome until late telophase. These include: nucleolin, fibrillarin, B23, p52, p66, p103, perichromin, peripherin and the Ki-67 antigen.

Because of their association with mitotic chromosomes or other components of a re-forming nucleus, any of these proteins may serve as, or contain, a potential chromosomal targeting sequence.

Interestingly, when anti-DNA antibodies were microinjected into dividing mammalian cells, they, unlike other macromolecules, did accumulate in the nuclei of the daughter cells after mitosis. This finding supports our claim that molecules that are not endogenous components of telophase chromosomes can nevertheless be targeted to newly forming nuclei through association with chromosomes.

A Cromosome Targeting Sgnal (CTS) is defined in this specification as a molecule that enhances localization of an associated compound such as a nucleic acid, protein, drug or transfection reagent, to within the nucleus of a dividing eukaryotic cell. Targeting of the compound to within the nucleus is not dependent on transport through a nuclear pore complex. The CTS can be a protein, peptide, protein fragment, lipid, antibody, antibody fragment, or a synthetic or natural molecule that interacts with mitotic chromosomes or other mitotic component such that the molecule is contained in the nucleus when the nuclear envelope reassembles at the end of mitosis.

The term nucleic acid, or polynucleotide, is a term of art that refers to a polymer containing at least two nucleotides. Natural nucleotides contain a deoxyribose (DNA) or ribose (RNA) group, a phosphate group, and a base. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. Nucleotides are the monomeric units of nucleic acid polymers and are linked together through the phosphate groups. Polynucleotides with less than 120 monomeric units are often called oligonucleotides. The term polynucleotide includes deoxyribonucleic acid (DNA) and ribonucleic acid (RNA). Natural polynucleotides have a ribose-phosphate backbone. An artificial or synthetic polynucleotide is any polynucleotide that is polymerized in vitro and contains the same or similar bases but may contain a backbone of a type other than the natural ribose-phosphate backbone. These backbones include, but are not limited to: PNAs (peptide nucleic acids), phosphorothioates, phosphorodiamidates, morpholinos, and other variants of the phosphate backbone of natural polynucleotides.

DNA may be in form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups. An anti-sense polynucleotide is a polynucleotide that interferes with the function of DNA and/or RNA. Interference may result in suppression of expression. The polynucleotide can also be a sequence whose presence or expression in a cell alters the expression or function of cellular genes or RNA. In addition, DNA and RNA may be single, double, triple, or quadruple stranded.

A delivered nucleic acid can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

A nucleic acid can be delivered to a cell to express an exogenous nucleotide sequence, to inhibit, eliminate, augment, or alter expression of an endogenous nucleotide sequence, or to affect a specific physiological characteristic not naturally associated with the cell. Nucleic acids may contain an expression cassette coded to express a whole or partial protein, or RNA. An expression cassette refers to a natural or recombinantly produced nucleic acid that is capable of expressing a gene(s). The term recombinant as used herein refers to a nucleic acid molecule that is comprised of segments of polynucleotide joined together by means of molecular biological techniques. The cassette contains the coding region of the gene of interest along with any other sequences that affect expression of the gene. A DNA expression cassette typically includes a promoter (allowing transcription initiation), and a sequence encoding one or more protein s. Optionally, the expression cassette may include, but is not limited to, transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include, but is not limited to, translation termination signals, a polyadenosine sequence, internal ribosome entry sites (IRES), and non coding sequences.

The nucleic acid may contain sequences that do not serve a specific function in the target cell but are used in the generation of the nucleic acid. Such sequences include, but are not limited to, sequences required for replication or selection of the nucleic acid in a host organism.

The terms naked nucleic acid and naked polynucleotide indicate that the nucleic acid or polynucleotide is not associated with a transfection reagent or other delivery vehicle that is required for the nucleic acid or polynucleotide to be delivered to the cell. A transfection reagent is a compound or compounds that bind(s) to or complex(es) with oligonucleotides and polynucleotides, and mediates their entry into cells. The transfection reagent also mediates the binding and internalization of oligonucleotides and polynucleotides into cells. Examples of transfection reagents include, but are not limited to, cationic lipids and liposomes, polyamines, calcium phosphate precipitates, histone proteins, polyethylenimine, and polylysine complexes. It has been shown that cationic proteins like histones and protamines, or synthetic cationic polymers like polylysine, polyarginine, polyornithine, DEAE dextran, polybrene, and polyethylenimine may be effective intracellular delivery agents, while small polycations like spermine are ineffective. Typically, the transfection reagent has a net positive charge that binds to the oligonucleotide's or polynucleotide's negative charge. The transfection reagent mediates binding of oligonucleotides and polynucleotides to cells via its positive charge (that binds to the cell membrane's negative charge) or via cell targeting signals that bind to receptors on or in the cell. For example, cationic liposomes or polylysine complexes have net positive charges that enable them to bind to DNA or RNA. Polyethylenimine, which facilitates gene transfer without additional treatments, probably disrupts endosomal function itself.

A nucleic acid can be used to modify the genomic or extrachromosomal DNA sequences. This can be achieved by delivering a nucleic acid that is expressed. Alternatively, the nucleic acid can effect a change in the DNA or RNA sequence of the target cell. This can be achieved by hybridization, multistrand nucleic acid formation, homologous recombination, gene conversion, or other yet to be described mechanisms.

The term gene generally refers to a nucleic acid sequence that comprises coding sequences necessary for the production of a therapeutic nucleic acid (e.g., ribozyme) or a polypeptide or precursor. The polypeptide can be encoded by a full length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term gene encompasses both cDNA and genomic forms of a gene. A genomic form or clone of a gene contains the coding region interrupted with non-coding sequences termed introns, intervening regions or intervening sequences. Introns are segments of a gene which are transcribed into nuclear RNA. Introns may contain regulatory elements such as enhancers. Introns are removed or spliced out from the nuclear or primary transcript; introns therefore are absent in the messenger RNA (mRNA) transcript. The mRNA functions during translation to specify the sequence or order of amino acids in a nascent polypeptide. The term non-coding sequences also refers to other regions of a genomic form of a gene including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotide) or at distant sites (more than 10,000 nucleotides). These non-coding sequences influence the level or rate of transcription and translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, and immunogenicity. One example of covalent modification of nucleic acid involves the action of LabellT reagents (Mirus Corporation, Madison, Wis.).

As used herein, the term gene expression refers to the process of converting genetic information encoded in a gene into RNA (e.g., mRNA, rRNA, tRNA, or snRNA) through transcription of a deoxyribonucleic gene (e.g., via the enzymatic action of an RNA polymerase), and for protein encoding genes, into protein through translation of mRNA. Gene expression can be regulated at many stages in the process. Up-regulation or activation refers to regulation that increases the production of gene expression products (i.e., RNA or protein), while down-regulation or repression refers to regulation that decrease production. Molecules (e.g., transcription factors) that are involved in up-regulation or down-regulation are often called activators and repressors, respectively.

Protein refers herein to a linear series of greater than 2 amino acid residues connected one to another via peptide bonds as in a polypeptide. A therapeutic effect of the protein in attenuating or preventing the disease state can be accomplished by the protein either staying within the cell, remaining attached to the cell in the membrane, or being secreted and dissociated from the cell where it can enter the general circulation and blood. Secreted proteins that can be therapeutic include hormones, cytokines, growth factors, clotting factors, anti-protease proteins (e.g., alpha1-antitrypsin), angiogenic proteins (e.g., vascular endothelial growth factor, fibroblast growth factors), anti-angiogenic proteins (e.g., endostatin, angiostatin), and other proteins that are present in the blood. Proteins on the membrane can have a therapeutic effect by providing a receptor for the cell to take up a protein or lipoprotein (e.g., low density lipoprotein receptor). Therapeutic proteins that stay within the cell (intracellular proteins) can be enzymes that clear a circulating toxic metabolite as in phenylketonuria. They can also cause a cancer cell to be less proliferative or cancerous (e.g., less metastatic), or interfere with the replication of a virus. Intracellular proteins can be part of the cytoskeleton (e.g., actin, dystrophin, myosins, sarcoglycans, dystroglycans) and thus have a therapeutic effect in cardiomyopathies and musculoskeletal diseases (e.g., Duchenne muscular dystrophy, limb-girdle disease). Other therapeutic proteins of particular interest to treating heart disease include polypeptides affecting cardiac contractility (e.g., calcium and sodium channels), inhibitors of restenosis (e.g., nitric oxide synthetase), angiogenic factors, and anti-angiogenic factors.

Polymer. A polymer is a molecule built up by repetitive bonding together of smaller units called monomers. Small polymer having 2 to about 80 monomers can be called oligomers. The polymer can be linear, branched network, star, comb, or ladder type. The polymer can be a homopolymer in which a single monomer is used or can be copolymer in which two or more monomers are used. Types of copolymers include alternating, random, block and graft.

The main chain of a polymer is composed of the atoms whose bonds are required for propagation of polymer length. The side chain of a polymer is composed of the atoms whose bonds are not required for propagation of polymer length.

To those skilled in the art, there are several categories of polymerization processes that can be utilized. The polymerization can be chain or step. This classification description is more often used than the previous terminology of addition and condensation polymerization. “Most step-reaction polymerizations are condensation processes and most chain-reaction polymerizations are addition processes” (M. P. Stevens Polymer Chemistry: An Introduction New York Oxford University Press 1990). Template polymerization can be used to form polymers from daughter polymers.

Cleavable polymers. For inhibitor complexes, the inhibitor must be dissociated from components of the complex in the cell in order for the inhibitor to be active. This dissociation may occur outside the cell, within cytoplasmic vesicles or organelles (i.e. endosomes), in the cytoplasm, or in the nucleus. We have developed bulk polymers prepared from disulfide bond containing co-monomers and cationic co-monomers to better facilitate this process. These polymers have been shown to condense polynucleotides, and to release the nucleotides after reduction of the disulfide bond. These polymers can be used to effectively complex with nucleic acids and can also protect the nucleic acid from nucleases during delivery to the liver and other organs. After delivery to the cells the polymers are reduced to monomers, effectively releasing the nucleic acid. For instance, the disulfide bonds may be reduced by glutathione which is present in higher concentrations inside the cell. Negatively charged polymers can be fashioned in a similar manner, allowing the condensed nucleic acid particle to be “recharged” with a cleavable anionic polymer resulting in a particle with a net negative charge that after reduction of disulfide bonds will release the nucleic acid. The reduction potential of the disulfide bond in the reducible co-monomer can be adjusted by chemically altering the disulfide bonds environment. Therefore one can construct particles whose release characteristics can be tailored so that the nucleic acid is released at the proper point in the delivery process.

Polyelectrolyte/polycation/polyanion. A polyelectrolyte, or polyion, is a polymer possessing more than one charge, i.e. the polymer contains groups that have either gained or lost one or more electrons. A polycation is a polyelectrolyte possessing net positive charge, for example poly-L, lysine hydrobromide. The polycation can contain monomer units that are charge positive, charge neutral, or charge negative, however, the net charge of the polymer must be positive. A polycation also can mean a non-polymeric molecule that contains two or more positive charges. A polyanion is a polyelectrolyte containing a net negative charge. The polyanion can contain monomer units that are charge negative, charge neutral, or charge positive, however, the net charge on the polymer must be negative. A polyanion can also mean a non-polymeric molecule that contains two or more negative charges. The term polyelectrolyte includes polycation, polyanion, zwitterionic polymers, and neutral polymers. The term zwitterionic refers to the product (salt) of the reaction between an acidic group and a basic group that are part of the same molecule.

Polymers have been used in research for the delivery of nucleic acids to cells. One of the several methods of nucleic acid delivery to the cells is the use of nucleic acid/polycation complexes. It has been shown that cationic proteins, like histones and protamines, or synthetic polymers, like polylysine, polyarginine, polyornithine, DEAE dextran, polybrene, and polyethylenimine, but not small polycations like spermine may be effective intracellular DNA delivery agents. Multivalent cations with a charge of three or higher have been shown to condense nucleic acid when 90% or more of the charges along the sugar-phosphate backbone are neutralized. The volume which one polynucleotide molecule occupies in a complex with polycations is lower than the volume of a free polynucleotide molecule. Polycations also provide attachment of polynucleotide to a cell surface. The polymer forms a cross-bridge between the polyanionic nucleic acid and the polyanionic surface of the cell. As a result, the mechanism of nucleic acid translocation to the intracellular space might be non-specific adsorptive endocytosis. Furthermore, polycations provide a convenient linker for attaching specific ligands to the complex. The nucleic acid/polycation complexes could then be targeted to specific cell types. Complex formation also protects against nucleic acid degradation by nucleases present in serum as well as in endosomes and lysosomes. Protection from degradation in endosomes/lysosomes is enhanced by preventing organelle acidification. Disruption of endosomal/lysosomal function may also be accomplished by linking endosomal or membrane disruptive agents to the polycation or complex.

A DNA-binding protein is a protein that associates with nucleic acid under conditions described in this application and forms a complex with nucleic acid with a high binding constant. The DNA-binding protein can be used in an effective amount in its natural form or a modified form for this process. An effective amount of the polycation is an amount that will allow delivery of the inhibitor to occur.

A non-viral vector is defined as a vector that is not assembled within an eukaryotic cell including non-viral inhibitor/polymer complexes, inhibitor with transfection enhancing compounds and inhibitor+amphipathic compounds.

Two molecules are combined, to form a complex through a process called complexation or complex formation, if they are in contact with one another through non covalent interactions such as, but not limited to, electrostatic interactions, hydrogen bonding interactions, and hydrophobic interactions. An interpolyelectrolyte complex is a non-covalent interaction between polyelectrolytes of opposite charge. A molecule is modified, through a process called modification, by a second molecule if the two become bonded through a covalent bond. That is, the two molecules form a covalent bond between an atom form one molecule and an atom from the second molecule resulting in the formation of a new single molecule. A chemical covalent bond is an interaction, bond, between two atoms in which there is a sharing of electron density.

Delivery of a biologically active compound means to transfer a biologically active compound from a container to near or within the outer cell membrane of a cell in the mammal or in vitro. The term transfection is used herein, in general, as a substitute for the term delivery, or, more specifically, the transfer of a nucleic acid from directly outside a cell membrane to within the cell membrane.

The process of delivering a nucleic acid to a cell has been commonly termed transfection or the process of transfecting and also it has been termed transformation. The term transfecting as used herein refers to the introduction of foreign nucleic acid or other biologically active compound into cells. The biologically active compound could be used to produce a change in a cell that can be therapeutic. The delivery of nucleic acid for therapeutic and research purposes is commonly called gene therapy. The delivery of nucleic acid can lead to modification of the genetic material present in the target cell. The term stable transfection or stably transfected generally refers to the introduction and integration of foreign nucleic acid into the genome of the transfected cell. The term stable transfectant refers to a cell which has stably integrated foreign nucleic acid into the genomic DNA. Stable transfection can also be obtained by using episomal vectors that are replicated during the eukaryotic cell division (e.g., plasmid DNA vectors containing a papilloma virus origin of replication, artificial chromosomes). The term transient transfection or transiently transfected refers to the introduction of foreign nucleic acid into a cell where the foreign nucleic acid does not integrate into the genome of the transfected cell. The foreign nucleic acid persists in the nucleus of the transfected cell. The foreign nucleic acid is subject to the regulatory controls that govern the expression of endogenous genes in the chromosomes. The term transient transfectant refers to a cell which has taken up foreign nucleic acid but has not integrated this nucleic acid.

A suicide gene encodes a protein product which, under appropriate conditions, is able to kill a cell in which the suicide gene is expressed. The suicide gene may be selected from the group comprising: herpes simplex virus thymidine kinase (HSV-TK), deoxycytitine kinase (dCK), and diphtheria toxin A.

Functional group. Functional groups include cell targeting signals, nuclear localization signals, compounds that enhance release of contents from endosomes or other intracellular vesicles (releasing signals), and other compounds that alter the behavior or interactions of the compound or complex to which they are attached.

Cell targeting signals are any signals that enhance the association of the biologically active compound with a cell. These signals can modify a biologically active compound such as drug or nucleic acid and can direct it to a cell location (such as tissue) or location in a cell (such as the nucleus) either in culture or in a whole organism. The signal may increase binding of the compound to the cell surface and/or its association with an intracellular compartment. By modifying the cellular or tissue location of the foreign gene, the function of the biologically active compound can be enhanced. The cell targeting signal can be, but is not limited to, a protein, peptide, lipid, steroid, sugar, carbohydrate, (non-expressing) polynucleic acid or synthetic compound. Cell targeting signals such as ligands enhance cellular binding to receptors. A variety of ligands have been used to target drugs and genes to cells and to specific cellular receptors. The ligand may seek a target within the cell membrane, on the cell membrane or near a cell. Binding of ligands to receptors typically initiates endocytosis. Ligands include agents that target to the asialoglycoprotein receptor by using asiologlycoproteins or galactose residues. Other proteins such as insulin, EGF, or transferrin can be used for targeting. Peptides that include the RGD sequence can be used to target many cells. Chemical groups that react with thiol, sulfhydryl, or disulfide groups on cells can also be used to target many types of cells. Folate and other vitamins can also be used for targeting. Other targeting groups include molecules that interact with membranes such as lipids, fatty acids, cholesterol, dansyl compounds, and amphotericin derivatives. In addition viral proteins could be used to bind cells.

After interaction of a compound or complex with the cell, other targeting groups can be used to increase the delivery of the biologically active compound to certain parts of the cell.

Nuclear localizing signals enhance the targeting of the pharmaceutical into proximity of the nucleus and/or its entry into the nucleus during interphase of the cell cycle. Such nuclear transport signals can be a protein or a peptide such as the SV40 large T antigen NLS or the nucleoplasmin NLS. These nuclear localizing signals interact with a variety of nuclear transport factors such as the NLS receptor (karyopherin alpha) which then interacts with karyopherin beta. The nuclear transport proteins themselves could also function as NLS's since they are targeted to the nuclear pore and nucleus. For example, karyopherin beta itself could target the DNA to the nuclear pore complex. Several peptides have been derived from the SV40 T antigen. Other NLS peptides have been derived from the hnRNP A1 protein, nucleoplasmin, c-myc, etc.

Many biologically active compounds, in particular large and/or charged compounds, are incapable of crossing biological membranes. In order for these compounds to enter cells, the cells must either take them up by endocytosis, i.e., into endosomes, or there must be a disruption of the cellular membrane to allow the compound to cross. In the case of endosomal entry, the endosomal membrane must be disrupted to allow for movement out of the endosome and into the cytoplasm. Either entry pathway into the cell requires a disruption of the cellular membrane. Compounds that disrupt membranes or promote membrane fusion are called membrane active compounds. These membrane active compounds, or releasing signals, enhance release of endocytosed material from intracellular compartments such as endosomes (early and late), lysosomes, phagosomes, vesicle, endoplasmic reticulum, golgi apparatus, trans golgi network (TGN), and sarcoplasmic reticulum. Release includes movement out of an intracellular compartment into the cytoplasm or into an organelle such as the nucleus. Releasing signals include chemicals such as chloroquine, bafilomycin or Brefeldin A1 and the ER-retaining signal (KDEL sequence), viral components such as influenza virus hemagglutinin subunit HA-2 peptides and other types of amphipathic peptides. The control of when and where the membrane active compound is active is crucial to effective transport. If the membrane active agent is operative in a certain time and place it would facilitate the transport of the biologically active compound across the biological membrane. If the membrane active compound is too active or active at the wrong time, then no transport occurs or transport is associated with cell rupture and cell death. Nature has evolved various strategies to allow for membrane transport of biologically active compounds including membrane fusion and the use of membrane active compounds whose activity is modulated such that activity assists transport without toxicity. Many lipid-based transport formulations rely on membrane fusion and some membrane active peptides' activities are modulated by pH. In particular, viral coat proteins are often pH-sensitive, inactive at neutral or basic pH and active under the acidic conditions found in the endosome.

Another functional group comprises compounds, such as polyethylene glycol, that decrease interactions between molecules and themselves and with other molecules. Such groups are useful in limiting interactions such as between serum factors and the molecule or complex to be delivered.

A covalent linkage is an attachment that provides a bond or spacer between two other groups (chemical moieties). The linkage may be electronically neutral, or may bear a positive or negative charge. The chemical moieties can be hydrophilic or hydrophobic. Preferred spacer groups include, but are not limited to C1-C12 alkyl, C1-C12 alkenyl, C1-C12 alkynyl, C6-C18 aralkyl, C6-C18 aralkenyl, C6-C18 aralkynyl, ester, ether, ketone, alcohol, polyol, amide, amine, polyglycol, polyether, polyamine, thiol, thio ether, thioester, phosphorous containing, and heterocyclic. The linkage may or may not contain one or more labile bonds.

A labile bond is a covalent bond that is capable of being selectively broken. That is, the labile bond may be broken in the presence of other covalent bonds without the breakage of other covalent bonds. For example, a disulfide bond is capable of being broken in the presence of thiols without cleavage of other bonds, such as carbon-carbon, carbon-oxygen, carbon-sulfur, carbon-nitrogen bonds, which may also be present in the molecule.

A labile linkage is a chemical compound that contains a labile bond and provides a link or spacer between two other groups. The groups that are linked may be chosen from compounds such as biologically active compounds, membrane active compounds, compounds that inhibit membrane activity, functional reactive groups, monomers, and cell targeting signals. The spacer group may contain chemical moieties chosen from a group that includes alkanes, alkenes, esters, ethers, glycerol, amide, saccharides, polysaccharides, and heteroatoms such as oxygen, sulfur, or nitrogen. The spacer may be electronically neutral, may bear a positive or negative charge, or may bear both positive and negative charges with an overall charge of neutral, positive or negative.

pH-labile refers to the selective breakage of a covalent bond under acidic conditions (pH<7). That is, the pH-labile bond may be broken under acidic conditions in the presence of other covalent bonds without their breakage.

A lipid is any of a diverse group of organic compounds that are insoluble in water, but soluble in organic solvents such as chloroform and benzene. Lipids contain both hydrophobic and hydrophilic sections. Lipids is meant to include complex lipids, simple lipids, and synthetic lipids. Complex lipids are the esters of fatty acids and include glycerides (fats and oils), glycolipids, phospholipids, and waxes. Simple lipids include steroids and terpenes. Synthetic lipids includes amides prepaired from fatty acids wherin the carboxylic acid has been converted to the amide, synthetic variants of complex lipids in which one or more oxygen atoms has been substitutied by another heteroatom (such as Nitrogen or Sulfur), and derivatives of simple lipids in which additional hydrophilic groups have been chemically attached. Synthetic lipids may contain one or more labile groups. Fats are glycerol esters of long-chain carboxylic acids. Hydrolysis of fats yields glycerol and a carboxylic acid—a fatty acid. Fatty acids may be saturated or unsaturated (contain one or more double bonds). Glycolipids are sugar containing lipids. The sugars are typically galactose, glucose or inositol. Phospolipids are lipids having both a phosphate group and one or more fatty acids (as esters of the fatty acid). The phosphate group may be bound to one or more additional organic groups. Waxes are any of various solid or semisolid substances generally being esters of fatty acids. Fatty acids are considered the hydrolysis product of lipids (fats, waxes, and phosphoglycerides)

### EXAMPLES

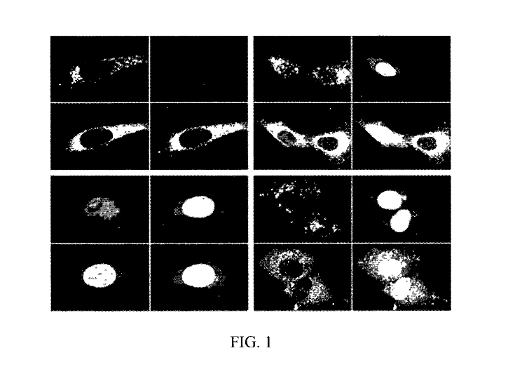

Exogenous DNA and large molecules are excluded from the nucleus following mitosis. In order to visualize the amount of pDNA in the nucleus and in the cytoplasm after mitosis, we injected a mixture of unlabeled pEYFP-Nuc plasmid, a fluorescently labeled pDNA and fluorescent dextran into HeLa cells. Following either cytoplasmic or nuclear microinjections, the physical location of the labeled DNA and dextran was detected both before and after mitosis. FIG. 1 demonstrates that in cells that had gone through mitosis both the dextran and the DNA became efficiently excluded from the newly formed nuclei. It was striking that both dextran and pDNA were excluded from the re-forming nuclei extremely efficiently, even if they had been injected into the nucleus. We hypothesize that, lacking a targeting mechanism to accumulate in the vicinity of the chromosomes, the fraction of delivered compound packaged into the newly formed nucleus is proportional to the volume of cytoplasm entrapped within the re-forming NE on the surface of telophase chromosomes. This observation fits well the estimation that <1% of the cytoplasmically delivered DNA reaches the nucleus, or remains in the nucleus, after mitosis.

Expression of microinjected DNA. We found that pDNA expressed several hundred-fold more efficiently when microinjected into the nucleus rather than into the cytoplasm. Table 1 shows the results in terms of the number of pEYFP-Nuc molecules injected per cell. In order to enable 50% of the cells to express EYFP-Nuc, it required injection of approximately 2000 copies into the cytoplasm. Conversely, injection of only 3 copies into the nucleus yielded 50% expression: a 700-fold difference. HeLa cells were injected with the indicated amount of pEYFP-Nuc plus 50 ng/μl inert carrier DNA to prevent loss of DNA from adsorption. The injection volume was 0.42 pl for cytoplasmic injection and 0.15 pl for nuclear injection. This volume corresponds to approximately 10% of the compartment volume. EYFP expression was assayed 20 hours after injection by fluorescent microscopy.

TABLE 1
Effect of pDNA concentration on expression levels in non-synchronized
HeLa cells. Cells were not scored for mitosis; during the 20 hours
incubation time approximately 70-80% of the cell population divided.

| | pEYFP molecules injected/cell | | % cells expressing YFP | |
|---|---|---|---|---|
| pEYFP-Nuc | cytoplasmic | nuclear | cytoplasmic | nuclear |
| (ng/μl) | injection | injection | injection | injection |
| 0.02 | 1.6 | 0.6 | 0.0 | 10.3 |
| 0.1 | 8 | 2.9 | 0.6 | 46.9 |
| 1 | 80 | 29 | 5.4 | 74 |
| 2 | 160 | 57 | 31 | 95 |
| 10 | 800 | 286 | 31 | 96 |
| 20 | 1600 | 571 | 41 | 100 |
| 25 | 2000 | 714 | 53 | 100 |

Effect of mitosis of expression of microinjected pDNA. We also evaluated individual injected cells, both for cell division and for marker gene expression. The data show that cells into which pDNA was injected cytoplasmically were able to express GFP without going through mitosis. Therefore some small fraction of the injected pDNA is able to enter the intact interphase nucleus. However, expression increased in cells that had gone through mitosis: from 28% to 50% after cytoplasmically injecting 10 ng/μl pEYFG-Nuc and from 50% to 90% after cytoplasmically injecting 1,000 ng/μl pEYFP-Nuc. However, expression levels never attained 100% in dividing cells, even when cytoplasmically injected with 1,000 ng/μl or 8×10⁴ copies of pEYFP-Nuc. Conversely, for nuclear injection of pDNA, a few hundred copies per nucleus results in 100% expression. Based on these observations we conclude that the amount of DNA that can enter the nucleus during mitosis is more than the amount entering through NPCs during interphase. However, even during mitosis, the amount of cytoplasmic DNA that gains access to the nucleus is less than 1%.

Attempts to enhance gene expression using NLS peptides. We have also experimented with promoting nuclear DNA uptake by the stable attachment of multiple copies of NLS peptides to linear DNA. We used a linear, <1 kb minimal expression cassette with a single biotin on one end. Streptavidin was covalently conjugated to either a 39 residue peptide (H-CKKKSSSDDEATADSQHSTPPKKKRKVEDPKDFPSELLS) [SEQ ID 1] containing the wild type SV40 NLS²⁷, or to a mutant version known to be transport deficient [SEQ ID 2].

Judged by SDS-PAGE the number of peptides per SA monomer was estimated to be 2. The conjugates were added to a linear, end-biotinylated and fluorescently labeled DNA, followed by microinjection into the cytoplasm of HeLa cells. Complexes with the functional NLS expressed the GFP marker gene 7 times more efficiently than complexes with the mutant NLS (an increase from 1.5% to 10.9%). Thus, using a stable bond between the linear DNA and multiple copies of a strong NLS, a 7-fold increase in expression efficiency could be obtained¹¹. However, the data also show that NLS-mediated uptake of DNA is size dependent, with nuclear targeting efficiency dropping dramatically for DNA molecules larger than 1 kb.

Size-dependence of NLS-mediated nuclear transport. Based on microinjection studies using fluorescently labeled linear DNA fragments of various sizes we observed that the efficiency of NLS-mediated nuclear transport was size-dependent. Fragments up to 500 bp efficiently accumulated in the nucleus of most injected cells. In contrast, a 1 kb fragment showed strong accumulation in the nucleus in only about 10% of the injected cells. Larger fragments, 2-3 kb in size, showed only faint nuclear accumulation in a small percentage of cells¹¹. These data suggest that the nuclear targeting of large DNA molecules can not be efficiently accomplished by NLS-mediated transfer through nuclear pore complexes. The concept of the present invention, that is targeting compounds to the nucleus during open mitosis, is void of this limitation.

Subcellular Location of Nuclear Antigens during Mitosis. HeLa cell cultures were enriched in mitotic cells by a double thymidine block. 9-10 h after releasing the cells from the block they were fixed with 4% formaldehyde and permeabilized with TritonX-100. An in vitro binding assay was performed with monoclonal antibodies (MAbs) against histone H1 (StressGene), Nup62, topoisomeraseIIβ, mitosin, and Ki-67 (Transduction Laboratories). The antibodies were detected with an Alexa488-labeled anti-mouse IgG (Alexa488-anti-MIgG) secondary antibody.

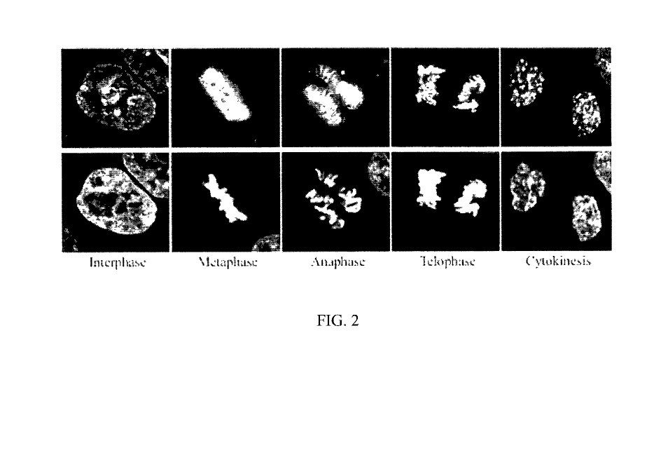

The anti Histone H1 MAb gives weak, finely punctate nuclear staining during interphase, and chromosomal staining during mitosis. The ends of the chromosomes stain more intensely than the centromeric regions. During interphase Nup62 shows a rim around the nucleus, with some additional weak, diffuse staining in the cytoplasm and nucleoplasm. During mitosis it is initially evenly dispersed throughout the cytoplasm, while fully excluded from the chromosomal volume. After anaphase, Nup62 starts to accumulate on the outside surface of the chromosome cluster, and by the end of cytokinesis, it again forms a rim around the new nucleus. The anti-mitosin MoAb yields grainy, non-nucleolar staining in the interphase nucleus. During mitosis most of mitosin is evenly dispersed in the cytoplasm, and a fraction of the antigen forms bright, small spots at the kinetochore of each chromosome. The interphase staining pattern of TopoisomeraseIIβ is very similar to Histone H1: a finely speckled pattern, including the nucleolar areas. During mitosis TopoIIβ is it was barely detectable, suggesting that the epitope to which the MAb binds is not accessible in the condensed mitotic chromosome. The anti-Ki-67 antibody shows intense peri-nucleolar staining during interphase, and re-distributes to a diffuse cloud around the chromosomes during metaphase and anaphase. As shown in FIG. 2, the signal is strong and comes exclusively from this peri-chromosomal sheath. There is no detectable fluorescence in the cytoplasm. The diffuse peri-chromosomal staining then becomes more distinctly co-localizing with chromosomes by telophase. After cytokinesis the antigen disengages from the chromosomes and migrates back to nucleoli.

Subcellular Distribution of MAbs after Microinjection. MAbs were diluted to 25 ng/μl in intracellular buffer (10 mM PIPES pH 7.2, 140 mM KCl, 1 mM MgCl₂) and were injected into the cytoplasm of HeLa cells. The cells were processed for microscopy 3-4h and 20-24 h after injection. The location of the injected MAb was determined by staining with Alexa488-anti-mouse IgG.

The staining pattern of the anti-Ki-67 MAb was similar to that observed in the in vitro binding assay. The MAb was strongly anchored to the chromosomes of mitotic cells with no detectable antigen left in the cytoplasm during mitosis (i.e., targeting is 100% effective). The anti-Ki-67 MAb then is a potential CTS in live cells. Surprisingly, nuclear entry of the MAb did not require mitosis, suggesting that the anti-Ki-67 MAb is actively transported along with the Ki-67 protein into the nucleus through NPCs. This MAb may therefore be used, not only as a CTS, but also as an NLS, enhancing nuclear localization of attached cargo/compound s during both interphase and mitosis.

The anti-TopoIIβ MAb did not enter interphase nuclei by 3-4 h but did accumulate in interphase nuclei after 20-24 h. The slow kinetics of nuclear localization in interphase nuclei may be due to slow transport of the epitope, topoisomeraseII, into the nucleus. Chromosome staining was not observed.

Neither antibody had apparent toxic effect at the 25 ng/μl concentration, ˜5×10⁴ IgG molecules per cell, used. Based on morphology the cells looked healthy and were dividing. Therefore, interference with normal cellular functions is unlikely for these MAbs, at this concentration.

Monoclonal versus Polyclonal Antibody. Polyclonal antibodies to the Ki-67 protein were generated. Protein-A purified antibodies from the polyclonal containing serum gave the same staining pattern as the MAb antibodies when tested on fixed cells. However, the polyclonal antibodies did not accumulate in the nuclei when microinjected into live cells. It is likely that the polyclonal antibodies bind to critical functional epitopes on Ki-67 or the Ki-67/polyclonal antibody complexes are too large to be transported. In mitotic cells, the polyclonal antibody showed a chromosomal staining pattern identical to the monoclonal antibody.

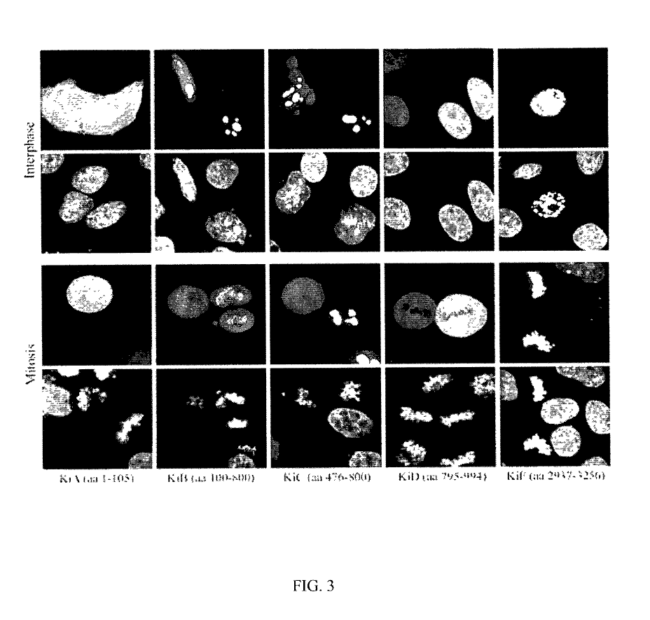

Mapping the Chromosome Targeting Domain of Ki-67. The primary sequence of the Ki-67 protein has been determined²⁸, and its domain structure has been partially characterized²⁹⁻³². However, none of the previous studies identified the domain responsible for directing Ki-67 to the peri-chromosomal sheath during mitosis. We therefore made a series of EYFP-fusions using various fragments of Ki-67. The fragments covered amino acid residues 1-105 (KiA), 100-800 (KiB), 476-800 (KiC), 795-994 (KiD), and 2937-3256 (KiF). The largest domain of the protein (KiE, amino acids 995-2936) contains 16 repeats of a 120 amino acid motif. We have not cloned this domain in full length, but we have looked at the subcellular distribution of a small fragment of it, which contains the 6th repeat motif (residues 1604-1725), with some flanking sequence on either end. This fragment did not accumulate in the nucleus during interphase, and did not bind to mitotic chromosomes (data not shown). The characteristic staining pattern of the other five domains in transiently transfected HeLa cells is shown in FIG. 3. The N-terminal KiA domain, also called the forkhead associated domain³¹, partially localizes to the nucleus in interphase cells, while some protein remains in the cytoplasm. During mitosis KiA shows diffuse cytoplasmic staining with scattered, bright spots (FIG. 3. KiA panels). KiB contains the protein's nucleolar localization signal. Both the full length KiB and its C-terminal half, KiC, accumulate in the nucleoli in interphase cells. During mitosis, they become evenly dispersed throughout the cytoplasm, with a weak peri-chromosomal accumulation visible in some cells (FIG. 3. KiB and KiC panels). The small domain between the nucleolar targeting domain and the 16 repeat domains, KiD accumulates in the nuclei very efficiently, but it is excluded from nucleoli. During mitosis KiD localization is similar to KiA: diffuse cytoplasmic staining with bright speckles (FIG. 3. KiD panels). The C-terminal KiF fragment, which had been shown to bind both DNA and the HP1 protein^{30,32}, co-localizes with the chromosomes during mitosis (FIG. 3. KiF panels). KiF, containing the C-terminal 320 residues, is sufficient for targeting the peri-chromosomal protein layer during mitosis. Targeting is just as efficient as with the full-length protein (FIG. 2). Thus, this truncated protein is a functional CTS, capable of targeting an attached fluorescent protein to mitotic chromosomes and into the newly formed nuclei of daughter cells.

Dominant Negative Effects of Overexpressed Ki-67 Fragments. Expression levels of the EYFP-Ki fusion proteins in transiently transfected cells were highly variable. In cells strongly over-expressing the Ki-67 fragments we observed obvious signs of toxicity: abnormal cell morphology, malformed nuclei, nuclear herniations, fragmented chromosomes, and floating dead cells. The frequency and severity of affected cells varied. Fragment KiB was by far the most detrimental, followed by KiC. Interphase cells marked with white arrows in FIG. 3. KiB and KiC panels have malformed nuclei with herniations and NE disruptions (leakage of nuclear material into the cytoplasm). In cultures transfected with EYFP-KiB only cells with very low expression levels survived (signal for KiB and KiC was enhanced relative to other pictures). KiA and KiD were well tolerated by the cells, even at high concentrations. KiF was also fairly well tolerated except at the very highest concentrations. Based on the ToPro3 DNA stain (FIG. 3, lower panels) the chromatin looked fragmented in these cells, and the DNA patches perfectly co-localized with extremely bright green KiF fluorescence (FIG. 3, Interphase cells; upper KiF panel). Similar toxic effects of this and other Ki-67 domains had been shown previously³⁰. Nevertheless, expression of apparently large amounts of this truncated Ki-67 protein do not appear to be toxic (FIG. 3 KiF). Production of recombinant KiF protein will allow quantitation of the tolerated concentration range tolerated by cells.

We have identified a MAb that binds to the Ki-67 antigen on the surface of mitotic chromosomes. The binding pattern, timing of binding, and abundant nature of the protein indicated that either the anti-Ki-67 antibody, the Ki67 protein itself, or domains of the Ki67 protein, could potentially be used as a CTS.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

### BIBLIOGRAPHY

1. Pante N, Aebi U. Molecular dissection of the nuclear pore complex. Critical Rev in Biochem Mol Biol. 1996 31:153-199.

2. Nigg E. Nucleocytoplasmic transport: signals, mechanisms and regulation. Nature. 1997 386:779-787.

3. Conti E, Izaurralde E. Nucleocytoplasmic transport enters the atomic age. Curr Opin Cell Biol. 2001 13:310-319.

4. Wilke M, Fortunati E, van den Broek M, Hoogeveen A T, Scholte B J. Efficacy of a peptide-based gene delivery system depends on mitotic activity. Gene Ther. 1996 3(12):1133-1142.

5. Mortimer I, Tam P, MacLachlan I, Graham R W, Saravolac E G, Joshi P B. Cationic lipid-mediated transfection of cells in culture requires mitotic activity. Gene Ther. 1999 6(3):403-411.

6. Brunner S, Sauer T, Carotta S, Cotten M, Saltik M, Wagner E. Cell cycle dependence of gene transfer by lipoplex, polyplex and recombinant adenovirus. Gene Ther. 2000 7(5):401-407.

7. Piolot T, Tramier M, Coppey M, Nicolas J C, Marechal V. Close but distinct regions of human herpesvirus 8 latency-associated nuclear antigen 1 are responsible for nuclear targeting and binding to human mitotic chromosomes. J Virol. 2001 75(8):3948-3959.

8. Marechal V, Dehee A, Chikhi-Brachet R, Piolot T, Coppey-Moisan M, Nicolas J C. Mapping EBNA-1 domains involved in binding to metaphase chromosomes. J Virol. 1999 73(5):4385-4392.

9. Swanson J A, McNeil P L. Nuclear reassembly excludes large macromolecules. Science. 1987 238:548-550.

10. Sebestyén M G, Ludtke J J, Bassik M C, Zhang G, Budker V, Lukhtanov E A, Hagstrom J E, Wolff J A. DNA vector chemistry: the covalent attachment of signal peptides to plasmid DNA. Nature Biotechnol. 1998 16:80-85.

11. Ludtke J J, Zhang G, Sebestyen M G, Wolff J A. A nuclear localization signal can enhance both the nuclear transport and expression of 1 kb DNA. J Cell Sci. 1999 112:2033-2041.

12. Subramanian A, Ranganathan P, Diamond S L. Nuclear targeting peptide scaffolds for lipofection of nondividing mammalian cells. Nat Biotechnol. 1999 17:873-877.

13. Chu A, Rassadi R, Stochaj U. Velcro in the nuclear envelope: LBR and LAPs. FEBS Lett. 1998 441:165-169.

14. Ye Q, Callebaut I, Pezhman A, Courvalin J C, Worman H J. Domain-specific interactions of human HP1-type chromodomain proteins and inner nuclear membrane protein LBR. J Biol Chem. 1997 272:14983-14989.

15. Vlcek S, Just H, Dechat T, Foisner R. Functional diversity of LAP2alpba and LAP2beta in postmitotic chromosome association is caused by an alpha-specific nuclear targeting domain. EMBO J. 1999 18:6370-6384.

16. Haraguchi T, Koujin T, Hayakawa T, Kaneda T, Tsutsumi C, Imamoto N, Akazawa C, Sukegawa J, Yoneda Y, Hiraoka Y. Live fluorescence imaging reveals early recruitment of emerin, LBR, RanBP2, and Nup153 to reforming functional nuclear envelopes. J Cell Sci. 2000 113:779-794.

17. Rolls M M, Stein P A, Taylor S S, Ha E, McKeon F, Rapoport T A. A visual screen of a GFP-fusion library identifies a new type of nuclear envelope membrane protein. J Cell Biol. 1999 146:29-44.

18. Drummond S, Ferrigno P, Lyon C, Murphy J, Goldberg M, Allen T, Smythe C, Hutchison C J. Temporal differences in the appearance of NEP-B78 and an LBR-like protein during Xenopus nuclear envelope reassembly reflect the ordered recruitment of functionally discrete vesicle types. J Cell Biol. 1999 144(2):225-240.

19. Ball A R, Yokomori K. The structural maintenance of chromosomes (SMC) family of proteins in mammals Chromosome Res. 2001 9(2):85-96.

20. Yang C H, Lambie E J, Snyder M. NuMA: an unusually long coiled-coil related protein in the mammalian nucleus. J Cell Biol. 1992 116(6): 1303-1317.

21. Ishimi Y, Komamura Y, You Z, Kimura H. Biochemical function of mouse minichromosome maintenance 2 protein. J Biol Chem. 1998 273(14):8369-8375.

22. Gitlits V M, Macaulay S L, Toh B H, Sentry J W. Novel human autoantibodies to phosphoepitopes on mitotic chromosomal autoantigens (MCAs). J Investig Med. 2000 48(3): 172-182.

23. Ludtke J J, Sebestyen M G and Wolff J A. The effect of cell division on the cellular dynamics of microinjected DNA and dextran. Mol Ther. (accepted for publication) 2002.

24. Yang L, Guan T, Gerace L. Integral membrane proteins of the nuclear envelope are dispersed throughout the endoplasmic reticulum during mitosis. J Cell Biol. 1997 137:1199-1210.

25. Cox, L S, Hutchison C J. Nuclear envelope assembly and disassembly. Subcell Biochem. 1995 22:263-325.

26. Georgatos S D, Theodoropoulos P A. Rules to remodel by: what drives nuclear envelope disassembly and reassembly during mitosis? Crit Rev Eukaryot Gene Expr. 1999 9:373-381.

27. Yoneda Y, Semba T, Kaneda Y, Noble R L, Matsuoka Y, Kurihara T, Okada Y, Imamoto N. A long synthetic peptide containing a nuclear localization signal and its flanking sequences of SV40 T-antigen directs the transport of IgM into the nucleus efficiently. Exp Cell Res. 1992 201:313-320.

28. Schluter C, Duchrow M, Wohlenberg C, Becker M H, Key G, Flad H D, Gerdes J. The cell proliferation-associated antigen of antibody Ki-67: a very large, ubiquitous nuclear protein with numerous repeated elements, representing a new kind of cell cycle-maintaining proteins. J Cell Biol. 1993 123:513-522.

29. Endl E, Gerdes J. The Ki-67 protein: fascinating forms and an unknown function. Exp Cell Res. 2000 257:231-237.

30. MacCallum D E, and Hall P A. The biochemical characterization of the DNA binding activity of pKi67. J Pathol. 2000 191:286-298.

31. Sueishi M, Takagi M, Yoneda Y. The forkhead-associated domain of Ki-67 antigen interacts with the novel kinesin-like protein Hklp2. J Biol Chem. 2000 275:28888-28892.

32. Scholzen T, Endl E, Wohlenberg C, van der Sar S, Cowell I G, Gerdes J, Singh P B. The Ki-67 protein interacts with members of the heterochromatin protein 1 (HP 1) family: a potential role in the regulation of higher-order chromatin structure. J Pathol. 2002 196:135-144.

We claim:

1. A compound for delivery to a cellular nucleus, comprising: a chromosome targeting signal constructed to associate with chromosomes during mitosis and be contained in the cell nucleus after mitosis.

2. The chromosome targeting signal of claim 1 wherein the chromosome targeting signal consists of a protein.

3. The protein of claim 2 wherein the protein is selected from the group consisting of protein fragments, peptides, synthetic proteins, synthetic peptides, and recombinant proteins.

4. The chromosome targeting signal of claim 1 wherein the chromosome targeting signal is selected from the group consisting of an antibody and antibody fragment.

5. The chromosome targeting signal of claim 1 wherein the chromosome targeting signal is a compound that interacts with a mitotic component.

6. A process for enhancing nuclear localization of a biologically active compound comprising: associating the biologically active compound with a chromosome targeting signal and delivering a resulting complex to a cell.

7. The process of claim 6 wherein the biologically active compound is a nucleic acid.

8. The process of claim 6 wherein the biologically active compound is a protein.

9. The process of claim 6 wherein the biologically active compound is a drug.

10. The process of claim 6 wherein the biologically active compound is in a complex.

11. The process of claim 6 wherein the association is a non-covalent interaction.

12. The process of claim 6 wherein the association is a covalent interaction.

13. The process of claim 12 wherein the covalent interaction is reversible.

14. The process of claim 12 wherein the covalent interaction is labile.

15. A process for nuclear localization of a compound, comprising: forming a compound consisting of a chromosome targeting signal and delivering the compound to a eukaryotic cell wherein the compound is contained within a cell nucleus after mitosis.

16. The process of claim 15 wherein the chromosome targeting signal comprises a nucleic acid.

17. The process of claim 15 wherein the cell is an actively growing cell.

18. The process of claim 17 wherein delivery of the compound results in cell death.

19. The process of claim 6 wherein the chromosome targeting signal associates with chromatin during mitosis.

20. The process of claim 15 wherein the chromosome targeting signal associates with chromatin during mitosis.
